(19) United States
(12) Patent Application Publication
(10) Pub. No.: US 2021/0393805 A1
(43) Pub. Date: Dec. 23, 2021

(54) PERFUSION-BASED DELIVERY OF RECOMBINANT AAV VECTORS FOR EXPRESSION OF SECRETED PROTEINS

(71) Applicant: University of Massachusetts, Boston, MA (US)

(72) Inventors: Alisha Gruntman, Worcester, MA (US); Terence Flotte, Holden, MA (US)

(73) Assignee: University of Massachusetts, Boston, MA (US)

(21) Appl. No.: 17/054,812

(22) PCT Filed: May 16, 2019
(86) PCT No.: PCT/US19/32593

§ 371 Date: Nov. 12, 2020

Related U.S. Application Data
(60) Provisional application No. 62/672,531, filed on May 16, 2018.

(51) Int. Cl. A61K 48/00; C12N 15/86

(52) U.S. Cl. CPC A61K 48/0075; C12N 2750/14143; C12N 15/86; A61K 48/0083

(57) ABSTRACT

In some aspects, the disclosure relates to methods and compositions for delivering a transgene to a subject. The disclosure is based, in part, on compositions (e.g., viral vectors, such as rAAV vectors) and methods of venous limb perfusion (VLP) that efficiently transduce muscle tissue and enhance serum concentrations of secreted transgenes.

### RELATED APPLICATIONS

This application is a national stage filing under 35 U.S.C. § 371 of international PCT application, PCT/US2019/032593, filed May 16, 2019, which claims priority under 35 U.S.C. § 119(e) to U.S. provisional patent application, U.S. Ser. No. 62/672,531, filed May 16, 2018, the entire contents of each of which are incorporated herein by reference.

### BACKGROUND

Efforts have been ongoing to develop muscle-based gene therapy with vectors that enable systemic secretion of certain transgenes (e.g. a rAAV1-AAT, the normal PiM version of the protein in AAT deficient patients). For example, delivery of rAAV1-CB-hAAT to the muscle of AAT-deficient patients in previous trials has proven to be safe and has demonstrated a dose-response relationship with the maximum levels achieved of approximately 300 nM, stable for 5 years after one set of 100 IM injections with a vector dose of 6×10¹² vg/kg in a volume of 135 ml. However, further increases in doses of gene therapy vectors are typically limited by the fact that the vector formulation cannot be concentrated further and that an increase in the volume of direct IM injection is not tolerable by patients.

### SUMMARY

Aspects of the disclosure relate to methods and compositions for delivery of a transgene (e.g., a therapeutic transgene) to a subject. The disclosure is based, in part, on methods for gene therapy administration that result in systemic secretion of transgene products (e.g., resulting in elevated serum levels of the transgene) in a subject.

Accordingly, in some aspects, the disclosure provides a method for delivering a transgene to a subject, the method comprising delivering a gene expression construct engineered to express one or more secreted gene products to an isolated limb of a subject, wherein circulation of blood through the vasculature of the isolated limb is interrupted, and wherein the delivery comprises the step of infusing a solution comprising the gene expression construct into a vein of the isolated limb.

In some embodiments, a gene expression construct comprises a viral vector. In some embodiments, a viral vector is a recombinant adeno-associated virus (AAV) vector, adenoviral (Ad), lentiviral vector (LV), or retroviral vector. In some embodiments, a viral vector is an rAAV vector. In some embodiments, between 1×10¹¹ and 1×10¹⁴ genome copies of a viral vector are delivered to a subject.

In some embodiments, a secreted gene product is an Alpha-1 antitrypsin (AAT) protein. In some embodiments, an AAT protein is a non-human primate AAT (e.g., monkey AAT, etc.). In some embodiments, an AAT protein is a human AAT, for example as represented by SEQ ID NO: 1.

In some embodiments, an expression construct comprises an isolated nucleic acid encoding the secreted gene product, optionally wherein the isolated nucleic acid sequence is operably linked to a promoter.

In some embodiments, a subject is a mammal, for example a human, non-human primate (e.g., monkey), rodent (e.g., mouse, rat, etc.), dog, or cat. In some embodiments, a subject is a human. In some embodiments a subject is characterized by a mutation in human AAT (e.g., as represented by Entrez Gene ID: 5265).

In some embodiments, an isolated limb is a lower extremity (e.g., a leg). In some embodiments, circulation of blood through the vasculature of an isolated limb is interrupted (or halted).

In some embodiments, delivery via venous limb perfusion (VLP) comprises the step of infusing a solution comprising the gene expression construct into a vein of the isolated limb. In some embodiments, a solution injected into the vein of the subject is between 10% and 50% of the lower extremity volume of the subject.

In some embodiments, delivery of the gene expression construct occurs over a period of between 5 minutes and 120 minutes.

### BRIEF DESCRIPTION OF DRAWINGS

FIG. 1 is a schematic depicting venous limb perfusion (VLP) and arterial balloon administration modalities.

FIGS. 2A-2F show images of the dosed limbs for each route of delivery. FIG. 2A shows Venous limb perfusion (VLP) dosed limb at the start of delivery. FIG. 2B shows VLP dosed limb 5 minutes into the vector infusion. FIG. 2C shows VLP dosed limb after all vector has been infused but tourniquet has not been released. FIG. 2D shows quadriceps dosing sight (note IM injection sights are denoted using a black marking pen). The animals body is to the left of the image and the knee to the right. FIG. 2E shows gastrocnemius dosing sight (note IM injection sights are denoted using a black marking pen). The animals knee is to the left of the image and the foot to the right. FIG. 2F shows Intra-arterial Push and Dwell (IAPD) dosed limb at the end of infusion before balloon catheters deflated.

FIG. 3 shows a Western blot analysis of AAV-CB-AATmyc transgene expression in injected animals.

FIG. 4A shows a histogram containing representative data relating to AAV-CB-AATmyc transgene expression in injected animals.

FIG. 4B shows RT-PCR data relating to AAV-CB-AATmyc transgene expression in injected animals after Day 60.

FIG. 5 is a schematic depicting a vector genome heatmap for different injection modalities (VLP, IAPD, IM) and different AAV constructs (AAV1-CB-AATmyc and AAV8-CB-AATmyc).

FIG. 6 shows data relating to quantification of total vector genome copy (Vg copies) number in lower extremity muscle tissue of injected animals. Quantification was estimated based on the volume of muscle tissue perfused and the nuclear density of muscle tissue.

FIGS. 7A-7C show serum creatine kinase and liver transaminase levels following rAAV Rhesus AAT-c-myc delivery. FIG. 7A shows serum levels of creatine kinase (CK) a marker of myocyte damage. FIG. 7B shows serum levels of alanine aminotransferase (ALT), a transaminase that increases in the serum following hepatocellular damage. FIG. 7C shows serum levels of aspartate aminotransferase (AST), a transaminase that increases in the serum following hepatocellular damage. IM=Intramuscular. ALT=Alanine amino transferase. AST=Aspartate aminotransferase. AAV1 and AAV8=AAV capsid type delivered. n=2 per group.

FIG. 8 shows IFNγ immune response to AAV1 capsid. Peripheral blood mononuclear cells collected prior to dosing and at necropsy (Day 60) were cultured 6 days before a 48 hour restimulation with AAV1 peptide pools. Comparing intramuscular (IM), intra-arterial push and dwell (IAPD) and hydrodynamic delivery (HPV) animals. Each graph represents a single animal. SFU: spot forming unit; * denotes a positive response; CD3/CD28: positive control; Control: media only negative control. Responses were considered positive when the number of spot-forming units (SFU) per million of cells were >50 and at least 3-fold higher than the control condition.

FIG. 9 shows IFNγ immune response to AAV8 capsid. Peripheral blood mononuclear cells collected prior to dosing and at necropsy (Day 60) were cultured 6 days before a 48 hour restimulation with AAV8 peptide pools. Comparing intramuscular (IM), intra-arterial push and dwell (IAPD) and hydrodynamic delivery (HPV) animals. Each graph represents a single animal. SFU: spot forming unit; * denotes a positive response; CD3/CD28: positive control; Control: media only negative control. Responses were considered positive when the number of spot-forming units (SFU) per million of cells were >50 and at least 3-fold higher than the control condition.

### DETAILED DESCRIPTION

In some aspects, the disclosure relates to methods and compositions for delivery of one or more transgenes (e.g., transgenes encoding secreted gene products) to a subject. The disclosure is based, in part, on the recognition that delivery of a high volume of solution comprising a gene expression construct to an isolated limb of a subject results in elevated levels of gene product in the serum of the subject.

### Transgene Delivery Methods

In some aspects, the disclosure provides a method for delivering a transgene to a subject, the method comprising delivering a gene expression construct engineered to express one or more secreted gene products to an isolated limb of a subject, wherein circulation of blood through the vasculature of the isolated limb is interrupted, and wherein the delivery comprises the step of infusing a solution comprising the gene expression construct into a vein of the isolated limb.

As used herein, a “gene expression construct” refers to an isolated nucleic acid or vector (e.g., plasmid, cosmid, bacmid, viral vector, etc.) that is engineered to express a transgene, such as a secreted gene product. Generally, a gene expression construct comprises a nucleic acid sequence encoding a gene product (e.g., a secreted gene product, e.g., a secreted protein) and one or more regulatory elements (e.g., a promoter sequence, enhancer sequence, Kozak sequence, polyA tail, etc.).

In some embodiments, a gene expression construct comprises a viral vector. Examples of viral vectors include but are not limited to recombinant adeno-associated virus (AAV) vectors, adenoviral (Ad) vectors, lentiviral vectors (LV), and retroviral vectors.

The adenovirus genome is a non-enveloped, large (36-kb) double-stranded DNA (dsDNA) molecule comprising multiple, heavily spliced transcripts. Adenoviruses have high packaging capacity (˜8 kilobases) and are able to target a broad range of dividing and non-dividing cells. Adenoviruses do not integrate into the host genome and thus only produce transient transgene expression in host cells. At either end of adenoviral genome are inverted terminal repeats (ITRs). Genes encoded by the adenoviral genome are divided into early (E1-E4) and late (L1-L5) transcripts. Most human adenoviral vectors are based on the Ad5 virus type, which uses the Coxsackie-Adenovirus Receptor to enter cells.

Retrovirus (most commonly, 7-retrovirus) is an RNA virus comprised of the viral genome and several structural and enzymatic proteins, including reverse transcriptase and integrase. Once inside a host cell, the retrovirus uses the reverse transcriptase to generate a DNA provirus from the viral genome. The integrase protein then integrates this provirus into the host cell genome for production of viral genomes encoding the nucleic acid(s) of interest. Retrovirus can package relatively high amounts of DNA (up to 8 kilobases), but are unable to infect non-dividing cells and insert randomly into the host cell genome.

Lentiviral vectors are derived from human immunodeficiency virus-1 (HIV-1). The lentiviral genome consists of single-stranded RNA that is reverse-transcribed into DNA and then integrated into the host cell genome. Lentiviruses can infect both dividing and non-dividing cells, making them attractive tools for gene therapy.

In some embodiments, a viral vector is a recombinant AAV (rAAV) vector. rAAV vectors and recombinant adeno-associated viruses (rAAVs) are described in further detail elsewhere in this disclosure.

Aspects of the disclosure relate to gene expression constructs engineered to express one or more secreted gene products. As used herein, “secreted gene product” refers to a molecule, such as a peptide, protein, etc., that is secreted from a cell (into an extracellular environment, such as blood, cerebrospinal fluid, interstitial space, stroma, etc.) after translation. Examples of secreted gene products include but are not limited to Alpha-1 antitrypsin (AAT) protein, secreted tumor suppressor proteins (e.g., IGFBP7, SRPX, etc.), SOD1, erythropoietin (EPO), insulin, interferon, etc. In some embodiments, a secreted gene product is not naturally secreted by a cell but is engineered to comprise a secretion signal sequence (e.g., a signal peptide) that results in secretion of the gene product from a cell.

In some embodiments, a secreted gene product is a protein. In some embodiments, a protein is Alpha-1 antitrypsin (AAT). In some embodiments, AAT is a non-human primate AAT (e.g., monkey AAT, etc.). In some embodiments, an AAT protein is a human AAT, for example as represented by SEQ ID NO: 1. Additional examples of secreted proteins include but are not limited to hormones (e.g., oxytocin, insulin, prostaglandins, steroids, etc.), enzymes (e.g., phospholipase enzymes, proteases, amylase, etc.), toxins (e.g., botulinum toxin, etc.), and antimicrobial peptides (e.g., dermcidin, indolicidin, beta-definsin 1, etc.).

In some aspects, the disclosure relates to methods that comprise a step of delivering a gene expression construct to an isolated limb of a subject. An “isolated” limb refers to a limb which has been manipulated in order to reduce (e.g., interfere with) or halt the flow of blood through the vasculature of the limb. For example, in some embodiments, an isolated limb is mechanically restrained (e.g., by a tourniquet, pressure cuff, etc.) at a location that restricts or cuts off circulatory flow to the portion of the limb that is distal (e.g., away from the point at which the limb connects to the trunk of a subject) to the location at which the limb has been mechanically restrained. In some embodiments, an isolated limb is further manipulated to remove blood from the vasculature (e.g., arteries, veins, capillaries, or any combination thereof) after the flow of blood to the limb has been interrupted or halted.

In some embodiments, delivery of a gene expression construct to a subject occurs after a limb has been isolated. In some embodiments, delivery of a gene expression construct comprises the step of infusing a solution comprising the gene expression construct into a vein of the isolated limb. Methods of infusing solutions into the vasculature of a subject are known in the art and include, for example, administration by inserting into a vein of a subject a cannula comprising a catheter connected to a container (e.g., IV infusion bag) which holds the solution to be infused.

A solution comprising a gene expression construct may vary in volume. In some embodiments, a solution injected into the vein of the subject is between 10% and 50% (e.g., 10%, 15%, 20%, 25%, 30%, 35%, 40%, 45%, or 50%) of the lower extremity volume of the subject.

In the context of viral vectors, the number of genome copies of a viral vector in a solution can vary. In some embodiments, between 1×10¹¹ and 1×10¹⁴ genome copies (e.g., 1×10¹¹, 1×10¹², 1×10¹³, 1×10¹⁴ genome copies) of a viral vector are delivered to a subject.

In some embodiments, delivery of the gene expression construct occurs over a period of between 5 minutes and 120 minutes (e.g., 5, 6, 7, 8, 9, 10, 15, 20, 30, 40, 50, 60, 75, 90, 120, or any number of minutes between 5 and 120).

### Alpha-1 Antitrypsin Deficiency

In some aspects, the disclosure relates to methods and compositions for secretion of Alpha-1 antitrypsin (AAT) into the serum of a subject. Alpha-1 antitrypsin (AAT) is a protein that functions as proteinase (protease) inhibitor. AAT is mainly produced in the liver, but functions primarily in the liver and the lungs. In some embodiments, an AAT protein is a non-mammalian primate AAT, for example as a protein comprising the sequence set forth in NCBI Reference Sequence Accession No. NP_001252946.1. In some embodiments, an AAT protein is a human AAT protein, for example a protein comprising the sequence set forth in Reference Sequence Accession No. NP_001121179.1, or SEQ ID NO: 1:

(SEQ ID NO: 1)
MPSSVSWGILLLAGLCCLVPVSLAEDPQGDAAQKTDTSHHDQDHPTFNK
ITPNLAEFAFSLYRQLAHQSNSTNIFFSPVSIATAFAMLSLGTKADTHD
EILEGLNFNLTEIPEAQIHEGFQELLRTLNQPDSQLQLTTGNGLFLSEG
LKLVDKFLEDVKKLYHSEAFTVNFGDTEEAKKQINDYVEKGTQGKIVDL
VKELDRDTVFALVNYIFFKGKWERPFEVKDTEEEDFHVDQVTTVKVPMM
KRLGMFNIQHCKKLSSWVLLMKYLGNATAIFFLPDEGKLQHLENELTHD
IITKFLENEDRRSASLHLPKLSITGTYDLKSVLGQLGITKVFSNGADLS
GVTEEAPLKLSKAVHKAVLTIDEKGTEAAGAMFLEAIPMSIPPEVKFNK
PFVFLMIEQNTKSPLFMGKVVNPTQK.

In some embodiments, an AAT protein is encoded by a mRNA sequence as set forth in Reference Sequence Accession No. NM_001127707.1, or SEQ ID NO: 2:

| | |
|---|---|
| 1 | ugggcaggaa cugggcacug ugcccagggc augcacugcc uccacgcagc aacccucaga |
| 61 | guccugagcu gaaccaagaa ggaggagggg gucgggccuc cgaggaaggc cuagccgcug |
| 121 | cugcugccag gaauuccagg uuggaggggc ggcaaccucc ugccagccuu caggccacuc |
| 181 | uccugugccu gccagaagag acagagcuug aggagagcuu gaggagagca ggaaagccuc |
| 241 | ccccguugcc ccucuggauc cacugcuuaa auacggacga ggacagggcc cugucuccuc |
| 301 | agcuucaggc accaccacug accugggaca gugaaucgac aaugccgucu ucugucucgu |
| 361 | ggggcauccu ccugcuggca ggccugugcu gccugguccc ugucucccug gcugaggauc |
| 421 | cccagggaga ugcugcccag aagacagaua caucccacca ugaucaggau cacccaaccu |
| 481 | ucaacaagau cacccccaac cuggcugagu ucgccuucag ccuauaccgc cagcuggcac |
| 541 | accaguccaa cagcaccaau aucuucuucu ccccagugag caucgcuaca gccuuugcaa |
| 601 | ugcucucccu ggggaccaag gcugacacuc acgaugaaau ccuggagggc cugaauuuca |
| 661 | accucacgga gauuccggag gcucagaucc augaaggcuu ccaggaacuc cuccguaccc |
| 721 | ucaaccagcc agacagccag cuccagcuga ccaccggcaa uggccuguuc cucagcgagg |
| 781 | gccugaagcu aguggauaag uuuuuggagg auguuaaaaa guuguaccac ucagaagccu |
| 841 | ucacugucaa cuucggggac accgaagagg ccaagaaaca gaucaacgau uacguggaga |
| 901 | aggguacuca agggaaaauu guggauuugg ucaaggagcu ugacagagac acaguuuuug |
| 961 | cucuggugaa uuacaucuuc uuuaaaggca aaugggagag acccuuugaa gucaaggaca |
| 1021 | ccgaggaaga ggacuuccac guggaccagg ugaccaccgu gaaggugccu augaugaagc |
| 1081 | guuuaggcau guuuaacauc cagcacugua agaagcuguc cagcugggug cugcugauga |
| 1141 | aauaccuggg caaugccacc gccaucuucu uccugccuga ugaggggaaa cuacagcacc |
| 1201 | uggaaaauga acucacccac gauaucauca ccaaguuccu ggaaaaugaa gacagaaggu |
| 1261 | cugccagcuu acauuuaccc aaacugucca uuacuggaac cuaugaucug aagagcgucc |
| 1321 | ugggucaacu gggcaucacu aaggucuuca gcaauggggc ugaccucucc ggggucacag |
| 1381 | aggaggcacc ccugaagcuc uccaaggccg ugcauaaggc ugugcugacc aucgacgaga |
| 1441 | aagggacuga agcugcuggg gccauguuuu uagaggccau acccaugucu aucccccccg |
| 1501 | aggucaaguu caacaaaccc uuugucuucu uaaugauuga acaaaauacc aagucucccc |
| 1561 | ucuucauggg aaaaguggug aaucccaccc aaaaauaacu gccucucgcu ccucaacccc |
| 1621 | uccccuccau cccuggcccc cucccuggau gacauuaaag aaggguugag cuggucccug |
| 1681 | ccugcaugug acuguaaauc ccucccaugu uuucucugag ucucccuuug ccugcugagg |
| 1741 | cuguaugugg gcuccaggua acagugcugu cuucgggccc ccugaacugu guucauggag |
| 1801 | caucuggcug gguaggcaca ugcugggcuu gaauccaggg gggacugaau ccucagcuua |
| 1861 | cggaccuggg cccaucuguu ucuggagggc uccagucuuc cuuguccugu cuuggagucc |
| 1921 | ccaagaagga aucacagggg aggaaccaga uaccagccau gaccccaggc uccaccaagc |
| 1981 | aucuucaugu cccccugcuc aucccccacu cccccccacc cagaguugcu cauccugcca |
| 2041 | gggcuggcug ugcccacccc aaggcugccc uccugggggc cccagaacug ccugaucgug |
| 2101 | ccguggccca guuuuguggc aucugcagca acacaagaga gaggacaaug uccuccucuu |
| 2161 | gacccgcugu caccuaacca gacucgggcc cugcaccucu caggcacuuc uggaaaauga |
| 2221 | cugaggcaga uucuuccuga agcccauucu ccauggggca acaaggacac cuauucuguc |
| 2281 | cuuguccuuc caucgcugcc ccagaaagcc ucacauaucu ccguuuagaa ucaggucccu |
| 2341 | ucuccccaga ugaagaggag ggucucugcu uuguuuucuc uaucuccucc ucagacuuga |
| 2401 | ccaggcccag caggccccag aagaccauua cccuauaucc cuucuccucc cuagucacau |
| 2461 | ggccauaggc cugcugaugg cucaggaagg ccauugcaag gacuccucag cuaugggaga |
| 2521 | ggaagcacau cacccauuga cccccgcaac cccucccuuu ccuccucuga gucccgacug |
| 2581 | gggccacaug cagccugacu ucuuugugcc uguugcuguc ccugcagucu ucagagggcc |
| 2641 | accgcagcuc cagugccacg gcaggaggcu guuccugaau agccccugug guaagggcca |
| 2701 | ggagaguccu uccauccucc aaggcccugc uaaaggacac agcagccagg aaguccccug |
| 2761 | ggccccuagc ugaaggacag ccugcucccu ccgucucuac caggaauggc cuuguccuau |
| 2821 | ggaaggcacu gccccauccc aaacuaaucu aggaaucacu gucuaaccac ucacugucau |
| 2881 | gaauguguac uuaaaggaug agguugaguc auaccaaaua gugauuucga uaguucaaaa |
| 2941 | uggugaaauu agcaauucua caugauucag ucuaaucaau ggauaccgac uguuucccac |
| 3001 | acaagucucc uguucucuua agcuuacuca cugacagccu uucacucucc acaaauacau |
| 3061 | uaaagauaug gccaucacca agcccccuag gaugacacca gaccugagag ucugaagacc |
| 3121 | uggauccaag uucugacuuu ucccccugac agcuguguga ccuucgugaa gucgccaaac |
| 3181 | cucucugagc cccagucauu gcuaguaaga ccugccuuug aguugguaug auguucaagu |
| 3241 | uagauaacaa aauguuuaua cccauuagaa cagagaauaa auagaacuac auuucuugca |

In some embodiments, compositions and methods described by the disclosure are useful for treating a subject having or suspected of having alpha-1 antitrypsin deficiency. As used herein the term, “alpha-1 antitrypsin deficiency” refers to a condition resulting from a deficiency of functional AAT in a subject. In some embodiments, a subject having an AAT deficiency produces insufficient amounts of alpha-1 antitrypsin. In some embodiments, a subject having an AAT deficiency produces a mutant AAT protein. In some embodiments, insufficient amounts of AAT or expression of mutant AAT protein results in damage to a subject's lung and/or liver. In some embodiments, the AAT deficiency leads to emphysema and/or liver disease. Typically, AAT deficiencies result from one or more genetic defects in the AAT gene. The one or more defects may be present in one or more copies (e.g., alleles) of the AAT gene in a subject. Typically, AAT deficiencies are most common among Europeans and North Americans of European descent. However, AAT deficiencies may be found in subjects of other descents as well.

Subjects (e.g., adult subjects) with severe AAT deficiencies are likely to develop emphysema. Onset of emphysema often occurs before age 40 in human subjects having AAT deficiencies. Smoking can increase the risk of emphysema in subjects having AAT deficiencies. Symptoms of AAT deficiency include shortness of breath, with and without exertion, and other symptoms commonly associated with chronic obstructive pulmonary disease (COPD). Other symptoms of AAT deficiencies include symptoms of severe liver disease (e.g., cirrhosis), unintentional weight loss, and wheezing. A physical examination may reveal a barrel-shaped chest, wheezing, or decreased breath sounds in a subject who has an AAT deficiency.

The following exemplary tests may assist with diagnosing a subject as having an AAT deficiency: an alpha-1 antitrypsin blood test, examination of arterial blood gases, a chest x-ray, a CT scan of the chest, genetic testing, and lung function test. In some cases, a subject having or suspected of having an AAT deficiency is subjected to genetic testing to detect the presence of one or more mutations in the AAT gene. In some embodiments, one or more of the mutations listed in Table 1 are detected in the subject.

In some cases, a physician may suspect that a subject has an AAT deficiency if the subject has emphysema at an early age (e.g., before the age of 40), emphysema without ever having smoked or without ever having been exposed to toxins, emphysema with a family history of an AAT deficiency, liver disease or hepatitis when no other cause can be found, liver disease or hepatitis and a family history of an AAT deficiency.

In some embodiments, alpha-1 antitrypsin deficiency can result in two distinct pathologic states: a lung disease which is primarily due to the loss of anti-protease function, and a liver disease due to a toxic gain of function of the mutant AAT protein (e.g., mutant PiZ-AAT).

### Isolated Nucleic Acids

In some aspects, the disclosure provides isolated nucleic acids, which may be rAAV vectors, useful for treating genetic disease. In some embodiments, the isolated nucleic acids comprise one or more regions that encode one or more inhibitory RNAs that target an endogenous mRNA of a subject. The isolated nucleic acids also typically comprise one or more regions that encode one or more exogenous mRNAs (e.g., one or more secreted gene products). The secreted gene products, for example protein(s), encoded by the one or more exogenous mRNA s may or may not be different in sequence composition than the protein(s) encoded by the one or more endogenous mRNAs. For example, the one or more endogenous mRNAs may encode a wild-type and mutant version of a particular protein, such as may be the case when a subject is heterozygous for a particular mutation, and the exogenous mRNA may encode a wild-type mRNA of the same particular protein. In this case, typically the sequence of the exogenous mRNA and endogenous mRNA encoding the wild-type protein are sufficiently different such that the exogenous mRNA is not targeted by the one or more inhibitory RNAs. This may be accomplished, for example, by introducing one or more silent mutations into the exogenous mRNA such that it encodes the same protein as the endogenous mRNA but has a different nucleic acid sequence. In this case, the exogenous mRNA may be referred to as “hardened.”

In another example, the one or more endogenous mRNAs may encode only mutant versions of a particular protein, such as may be the case when a subject is homozygous for a particular mutation, and the exogenous mRNA may encode a wild-type mRNA of the same particular protein. In this case, the sequence of the exogenous mRNA may be hardened as described above, or the one or more inhibitory RNAs may be designed to discriminate the mutated endogenous mRNA from the exogenous mRNA.

In some cases, the isolated nucleic acids typically comprise a first region that encodes one or more first inhibitory RNAs (e.g., miRNAs) comprising a nucleic acid having sufficient sequence complementary with an endogenous mRNA of a subject to hybridize with and inhibit expression of the endogenous mRNA, in which the endogenous mRNA encodes a first protein. The isolated nucleic acids also typically include a second region encoding an exogenous mRNA that encodes a second protein (e.g., a secreted gene product), in which the second protein has an amino acid sequence that is at least 85% identical to the first protein, in which the one or more first inhibitory RNAs do not comprise a nucleic acid having sufficient sequence complementary to hybridize with and inhibit expression of the exogenous mRNA. For example, the first region may be positioned at any suitable location. The first region may be positioned within an untranslated portion of the second region. The first region may be positioned in any untranslated portion of the nucleic acid, including, for example, an intron, a 5′ or 3′ untranslated region, etc.

In some cases, it may be desirable to position the first region upstream of the first codon of the exogenous mRNA. For example, the first region may be positioned between the first codon of the exogenous mRNA and 2000 nucleotides upstream of the first codon. The first region may be positioned between the first codon of the exogenous mRNA and 1000 nucleotides upstream of the first codon. The first region may be positioned between the first codon of the exogenous mRNA and 500 nucleotides upstream of the first codon. The first region may be positioned between the first codon of the exogenous mRNA and 250 nucleotides upstream of the first codon. The first region may be positioned between the first codon of the exogenous mRNA and 150 nucleotides upstream of the first codon.

In some cases, the first region may be positioned downstream of a portion of the second region encoding the poly-A tail of the exogenous mRNA. The first region may be between the last codon of the exogenous mRNA and a position 2000 nucleotides downstream of the last codon. The first region may be between the last codon of the exogenous mRNA and a position 1000 nucleotides downstream of the last codon. The first region may be between the last codon of the exogenous mRNA and a position 500 nucleotides downstream of the last codon. The first region may be between the last codon of the exogenous mRNA and a position 250 nucleotides downstream of the last codon. The first region may be between the last codon of the exogenous mRNA and a position 150 nucleotides downstream of the last codon.

The nucleic acid may also comprise a third region encoding a one or more second inhibitory RNAs (e.g., miRNAs) comprising a nucleic acid having sufficient sequence complementary to hybridize with and inhibit expression of the endogenous mRNA. As with the first region, the third region may be positioned at any suitable location. For example, the third region may be positioned in an untranslated portion of the second region, including, for example, an intron, a 5′ or 3′ untranslated region, etc. The third region may be positioned upstream of a portion of the second region encoding the first codon of the exogenous mRNA. The third region may be positioned downstream of a portion of the second region encoding the poly-A tail of the exogenous mRNA. In some cases, when the first region is positioned upstream of the first codon, the third region is positioned downstream of the portion of the second region encoding the poly-A tail of the exogenous mRNA, and vice versa.

In some cases, the first region and third regions encode the same set of one or more inhibitory RNAs (e.g., miRNAs). In other cases, the first region and third regions encode a different set of one or more inhibitory RNAs (e.g., miRNAs). In some cases, the one or more inhibitory RNAs (e.g., miRNAs) encoded by the first region target one or more of the same genes as the one or more inhibitory RNAs (e.g., miRNAs) encoded by the third region. In some cases, the one or more inhibitory RNAs (e.g., miRNAs) encoded by the first region do not target any of the same genes as the one or more inhibitory RNAs (e.g., miRNAs) encoded by the third region. It is to be appreciated that inhibitory RNAs (e.g., miRNAs) which target a gene have sufficient complementarity with the gene to bind to and inhibit expression (e.g., by degradation or inhibition of translation) of the corresponding mRNA.

The first and third regions may also encode a different number of inhibitory RNAs (e.g., miRNAs). For example, the first region and third regions may independently encode 1, 2, 3, 4, 5, 6 or more inhibitory RNAs (e.g., miRNAs). The first and third regions are not limited to comprising any one particular inhibitory RNA, and may include, for example, an miRNA, an shRNA, a TuD RNA, a microRNA sponge, an antisense RNAs, a ribozyme, an aptamer, or other appropriate inhibitory RNA. In some cases, the first region and/or third region comprises one or more miRNAs.

As disclosed herein, the second protein may have an amino acid sequence that is at least 85% identical to the first protein. Accordingly, the second protein may have an amino acid sequence that is at least 88%, at least 90%, at least 95%, at least 98%, at least 99% or more identical to the first protein. In some case, the second protein differs from the first protein by 1, 2, 3, 4, 5, 6, 7, 8, 9, 10 or more amino acids. In some cases, one or more of the differences between the first protein and second protein are conservative amino acid substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Accordingly, conservative amino acid substitutions may provide functionally equivalent variants, or homologs of an endogenous protein.

It should be appreciated that in some cases the second protein may be a marker protein (e.g., a fluorescent protein, a fusion protein, a tagged protein, etc.). Such constructs may be useful, for example, for studying the distribution of the encoded proteins within a cell or within a subject and are also useful for evaluating the efficiency of rAAV targeting and distribution in a subject.

In some embodiments of the isolated nucleic acids, the first protein (e.g., secreted gene product) is alpha-1 antitrypsin (AAT) protein. An exemplary sequence of a wild-type AAT is provided at SEQ ID NO: 1. In some embodiments, the endogenous mRNA may comprise the RNA sequence specified by the sequence set forth in SEQ ID NO: 2, as disclosed in PCT Publication WO2012/145624, the entire contents of which are incorporated herein by reference. In some cases, the AAT protein is a human AAT protein. The AAT protein may have a sequence as set forth in SEQ ID NO: 1 or one or more mutations thereof as identified in Table 1. The exogenous mRNA (e.g., secreted gene product) may have one or more silent mutations compared with the endogenous mRNA. The exogenous mRNA sequence may or may not encode a peptide tag (e.g., a myc tag, a His-tag, etc.) linked to the encoded protein. Often, in a construct used for clinical purposes, the exogenous mRNA sequence does not encode a peptide tag linked to the encoded protein.

As described further below, the isolated nucleic acids may comprise an inverted terminal repeats (ITR) of an AAV serotypes selected from the group consisting of: AAV1, AAV2, AAV5, AAV6, AAV6.2, AAV7, AAV8, AAV9, AAV10, AAV11 and variants thereof. The isolated nucleic acids may also include a promoter operably linked with the one or more first inhibitory RNAs, the exogenous mRNA, and/or the one or more second inhibitory RNAs. The promoter may be tissue-specific promoter, a constitutive promoter or inducible promoter.

TABLE 1
Mutations in Human AAT - Entrez Gene ID: 5265

| Chr. | mRNA | dbSNP rs# | | dbSNP | Protein | Codon | Amino acid |
|---|---|---|---|---|---|---|---|
| position | position | cluster id | Function | allele | residue | position | position |
| 94844794 | 1822 | rs78787657 | missense | A | Lys [K] | 1 | 417 |
| | | | contig reference | C | Gln [Q] | 1 | 417 |
| 94844797 | 1819 | rs3191200 | missense | C | Pro [P] | 1 | 416 |
| | | | contig reference | A | Thr [T] | 1 | 416 |
| 94844842 | 1774 | rs17850837 | missense | A | Lys [K] | 1 | 401 |
| | | | contig reference | C | Gln [Q] | 1 | 401 |
| 94844843 | 1773 | rs1303 | missense | C | Asp [D] | 3 | 400 |
| | | | contig reference | A | Glu [E] | 3 | 400 |
| 94844855 | 1761 | rs13170 | synonymous | T | Phe [F] | 3 | 396 |
| | | | contig reference | C | Phe [F] | 3 | 396 |
| 94844866 | 1750 | rs61761869 | missense | T | Ser [S] | 1 | 393 |
| | | | contig reference | C | Pro [P] | 1 | 393 |
| 94844887 | 1729 | rs12233 | missense | T | Ser [S] | 1 | 386 |
| | | | contig reference | C | Pro [P] | 1 | 386 |
| 94844912 | 1704 | rs28929473 | missense | T | Phe [F] | 3 | 377 |
| | | | contig reference | A | Leu [L] | 3 | 377 |
| 94844926 | 1690 | rs12077 | missense | T | Trp [W] | 1 | 373 |
| | | | contig reference | G | Gly [G] | 1 | 373 |
| 94844942 | 1674 | rs1050520 | synonymous | G | Lys [K] | 3 | 367 |
| | | | contig reference | A | Lys [K] | 3 | 367 |
| 94844947 | 1669 | rs28929474 | missense | A | Lys [K] | 1 | 366 |
| | | | contig reference | G | Glu [E] | 1 | 366 |
| 94844954 | 1662 | rs1050469 | synonymous | G | Thr [T] | 3 | 363 |
| | | | contig reference | C | Thr [T] | 3 | 363 |
| 94844957 | 1659 | rs1802961 | synonymous | T | Leu [L] | 3 | 362 |
| | | | contig reference | G | Leu [L] | 3 | 362 |
| 94844959 | 1657 | rs1131154 | missense | A | Met [M] | 1 | 362 |
| | | | contig reference | C | Leu [L] | 1 | 362 |
| 94844960 | 1656 | rs13868 | synonymous | A | Val [V] | 3 | 361 |
| | | | contig reference | G | Val [V] | 3 | 361 |
| 94844961 | 1655 | rs1131139 | missense | C | Ala [A] | 2 | 361 |
| | | | contig reference | T | Val [V] | 2 | 361 |
| 94844962 | 1654 | rs72555357 | frame shift | | | 1 | 361 |
| | | | contig reference | G | Val [V] | 1 | 361 |
| 94844965 | 1651 | rs1802959 | missense | A | Thr [T] | 1 | 360 |
| | | | contig reference | G | Ala [A] | 1 | 360 |
| 94844972 | 1644 | rs10427 | synonymous | C | Val [V] | 3 | 357 |
| | | | contig reference | G | Val [V] | 3 | 357 |
| 94844975 | 1641 | rs9630 | synonymous | T | Ala [A] | 3 | 356 |
| | | | contig reference | C | Ala [A] | 3 | 356 |
| 94844977 | 1639 | rs67216923 | frame shift | | | 1 | 356 |
| | | | frame shift | (15 bp) | | 1 | 356 |
| | | | contig reference | G | Ala [A] | 1 | 356 |
| 94845814 | 1625 | rs72555374 | frame shift | | | 2 | 351 |
| | | | contig reference | T | Leu [L] | 2 | 351 |
| 94845845 | 1594 | rs28929471 | missense | A | Asn [N] | 1 | 341 |
| | | | contig reference | G | Asp [D] | 1 | 341 |
| 94845893 | 1546 | rs1802962 | missense | T | Cys [C] | 1 | 325 |
| | | | contig reference | A | Ser [S] | 1 | 325 |
| 94845902 | 1537 | rs55704149 | missense | T | Tyr [Y] | 1 | 322 |
| | | | contig reference | G | Asp [D] | 1 | 322 |
| 94845914 | 1525 | rs117001071 | missense | T | Ser [S] | 1 | 318 |
| | | | contig reference | A | Thr [T] | 1 | 318 |
| 94845917 | 1521 | rs35624994 | frame shift | | Ser [S] | 3 | 316 |
| | | | frame shift | C | Ser [S] | 3 | 316 |
| | | | contig reference | CA | Ser [S] | 3 | 316 |
| 94847218 | 1480 | rs1802963 | nonsense | T | xxx [X] | 1 | 303 |
| | | | contig reference | G | Glu [E] | 1 | 303 |
| 94847262 | 1436 | rs17580 | missense | T | Val [V] | 2 | 288 |
| | | | contig reference | A | Glu [E] | 2 | 288 |
| 94847285 | 1413 | rs1049800 | synonymous | C | Asp [D] | 3 | 280 |
| | | | contig reference | T | Asp [D] | 3 | 280 |
| 94847306 | 1392 | rs2230075 | synonymous | T | Thr [T] | 3 | 273 |
| | | | contig reference | C | Thr [T] | 3 | 273 |
| 94847351 | 1347 | rs34112109 | synonymous | A | Lys [K] | 3 | 258 |
| | | | contig reference | G | Lys [K] | 3 | 258 |
| 94847357 | 1341 | rs8350 | missense | G | Trp [W] | 3 | 256 |
| | | | contig reference | T | Cys [C] | 3 | 256 |
| 94847386 | 1312 | rs28929470 | missense | T | Cys [C] | 1 | 247 |
| | | | contig reference | C | Arg [R] | 1 | 247 |
| 94847407 | 1291 | rs72552401 | missense | A | Met [M] | 1 | 240 |
| | | | contig reference | G | Val [V] | 1 | 240 |
| 94847415 | 1283 | rs6647 | missense | C | Ala [A] | 2 | 237 |
| | | | contig reference | T | Val [V] | 2 | 237 |
| 94847452 | 1246 | rs11558264 | missense | C | Gln [Q] | 1 | 225 |
| | | | contig reference | A | Lys [K] | 1 | 225 |
| 94847466 | 1232 | rs11558257 | missense | T | Ile [I] | 2 | 220 |
| | | | contig reference | G | Arg [R] | 2 | 220 |
| 94847475 | 1223 | rs11558265 | missense | C | Thr [T] | 2 | 217 |
| | | | contig reference | A | Lys [K] | 2 | 217 |
| 94849029 | 1119 | rs113813309 | synonymous | T | Asn [N] | 3 | 182 |
| | | | contig reference | C | Asn [N] | 3 | 182 |
| 94849053 | 1095 | rs72552402 | synonymous | T | Thr [T] | 3 | 174 |
| | | | contig reference | C | Thr [T] | 3 | 174 |
| 94849061 | 1087 | rs112030253 | missense | A | Arg [R] | 1 | 172 |
| | | | contig reference | G | Gly [G] | 1 | 172 |
| 94849109 | 1039 | rs78640395 | nonsense | T | xxx [X] | 1 | 156 |
| | | | contig reference | G | Glu [E] | 1 | 156 |
| 94849140 | 1008 | rs11558263 | missense | A | Arg [R] | 3 | 145 |
| | | | contig reference | C | Ser [S] | 3 | 145 |
| 94849151 | 997 | rs20546 | synonymous | T | Leu [L] | 1 | 142 |
| | | | contig reference | C | Leu [L] | 1 | 142 |
| 94849160 | 988 | rs11558261 | missense | A | Ser [S] | 1 | 139 |
| | | | contig reference | G | Gly [G] | 1 | 139 |
| 94849201 | 947 | rs709932 | missense | A | His [H] | 2 | 125 |
| | | | contig reference | G | Arg [R] | 2 | 125 |
| 94849228 | 920 | rs28931572 | missense | A | Asn [N] | 2 | 116 |
| | | | contig reference | T | Ile [I] | 2 | 116 |
| 94849303 | 845 | rs28931568 | missense | A | Glu [E] | 2 | 91 |
| | | | contig reference | G | Gly [G] | 2 | 91 |
| 94849325 | 823 | rs111850950 | missense | A | Thr [T] | 1 | 84 |
| | | | contig reference | G | Ala [A] | 1 | 84 |
| 94849331 | 817 | rs113817720 | missense | A | Thr [T] | 1 | 82 |
| | | | contig reference | G | Ala [A] | 1 | 82 |
| 94849345 | 803 | rs55819880 | missense | T | Phe [F] | 2 | 77 |
| | | | contig reference | C | Ser [S] | 2 | 77 |
| 94849364 | 784 | rs11575873 | missense | C | Arg [R] | 1 | 71 |
| | | | contig reference | A | Ser [S] | 1 | 71 |
| 94849381 | 767 | rs28931569 | missense | C | Pro [P] | 2 | 65 |
| | | | contig reference | T | Leu [L] | 2 | 65 |
| 94849388 | 760 | rs28931570 | missense | T | Cys [C] | 1 | 63 |
| | | | contig reference | C | Arg [R] | 1 | 63 |
| 94849466 | 682 | rs11558262 | missense | G | Ala [A] | 1 | 37 |
| | | | contig reference | A | Thr [T] | 1 | 37 |
| 94849492 | 656 | rs11558259 | missense | G | Arg [R] | 2 | 28 |
| | | | contig reference | A | Gln [Q] | 2 | 28 |
| 94849548 | 600 | rs11558260 | synonymous | T | Ile [I] | 3 | 9 |
| | | | contig reference | C | Ile [I] | 3 | 9 |
| | | | start codon | | | | 1 |

### METHODS OF USE

The invention also provides methods for expressing alpha 1-antitrypsin (AAT) protein in a subject (e.g., where the expressed AAT protein is secreted into the serum of the subject). Typically, the subject has or suspected of having an AAT deficiency. The methods typically involve administering to a subject an effective amount of a recombinant Adeno-Associated Virus (rAAV) harboring any of the isolated nucleic acids disclosed herein. In general, the “effective amount” of a rAAV refers to an amount sufficient to elicit the desired biological response. As will be appreciated by those of ordinary skill in this art, the effective amount of the recombinant AAV of the invention varies depending on such factors as the desired biological endpoint, the pharmacokinetics of the expression products, the condition being treated, the mode of administration, and the subject. Typically, the rAAV is administered with a pharmaceutically acceptable carrier.

The subject may have a mutation in an AAT gene. The mutation may result in decreased expression of wild-type (normal) AAT protein. The subject may be homozygous for the mutation. The subject may be heterozygous for the mutation. The mutation may be a missense mutation. The mutation may be a nonsense mutation. The mutation may be a mutation listed in Table 1. The mutation may result in expression of a mutant AAT protein. The mutant protein may be a gain-of-function mutant or a loss-of-function mutant. The mutant AAT protein may be incapable of inhibiting protease activity. The mutant AAT protein may fail to fold properly. The mutant AAT protein may result in the formation of protein aggregates. The mutant AAT protein may result in the formation of intracellular AAT globules. The mutation may result in a glutamate to lysine substitution at amino acid position 366 according to the amino acid sequence set forth as SEQ ID NO: 1. The methods may also involve determining whether the subject has a mutation. Accordingly the methods may involve obtaining a genotype of the AAT gene in the subject.

In some cases, after administration of the rAAV the level of expression of the first protein and/or second protein is determined in the subject. The administration may be performed on one or more occasions. When the administration is performed on one or more occasions, the level of the first protein and/or the level of the second protein in the subject are often determined after at least one administration. In some cases, the serum level of the secreted gene product (e.g., AAT protein) in the subject is increased by at least 20%, 30%, 40%, 50%, 60%, 70%, 80%, 85%, 90%, 100%, or more than 100% (e.g., 200%, 300%, 500%, etc.) following administration of the rAAV. In some embodiments, expression level of the secreted gene product is measured with respect to (e.g., relative to) a subject that has not been administered the rAAV. In some embodiments, expression level of the secreted gene product is measured with respect to (e.g., relative to) a subject that has been administered an rAAV encoding the same secreted gene product by a method other and a method as described by the disclosure (e.g., via IM delivery, etc.).

The increase in the level of the secreted gene product (e.g., AAT protein) may be sustained for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or more. In some cases, after 7 weeks of administration of the rAAV, the serum level of the secreted gene product is increased at least 50% compared with the serum level of the corresponding endogenous protein (e.g., level of endogenous AAT of the subject) prior to administration of the rAAV.

In some instances, after administration of the rAAV at least one clinical outcome parameter associated with the AAT deficiency is evaluated in the subject. Typically, the clinical outcome parameter evaluated after administration of the rAAV is compared with the clinical outcome parameter determined at a time prior to administration of the rAAV to determine effectiveness of the rAAV. Often an improvement in the clinical outcome parameter after administration of the rAAV indicates effectiveness of the rAAV. Any appropriate clinical outcome parameter may be used. Typically, the clinical outcome parameter is indicative of the one or more symptoms of an AAT deficiency. For example, the clinical outcome parameter may be selected from the group consisting of: serum levels of AAT, serum levels of AST, serum levels of ALT, presence of inflammatory foci, breathing capacity, cough frequency, phlegm production, frequency of chest colds or pneumonia, and tolerance for exercise. Intracellular AAT globules or inflammatory foci are evaluated in tissues effected by the AAT deficiency, including, for example, lung tissue or liver tissue.

### Recombinant AAVs

In some aspects, the disclosure provides isolated AAVs. As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, a rAAV having a capsid appropriate for the tissue being targeted can be selected. In some embodiments, the rAAV comprises a capsid protein having an amino acid sequence corresponding to any one of AAV1, AAV2, AAV3, AAV4, AAV5, AAV6, AAV7, AAV8, AAV9, AAV10, AAV11 and variants thereof. The recombinant AAVs typically harbor an isolated nucleic acid (e.g., gene expression construct) of the disclosure.

Methods for obtaining recombinant AAVs having a desired capsid protein are well known in the art (See, for example, US 2003/0138772, the contents of which are incorporated herein by reference in their entirety). AAVs capsid protein that may be used in the rAAVs of the invention a include, for example, those disclosed in G. Gao, et al., J. Virol, 78(12):6381-6388 (June 2004); G. Gao, et al, Proc Natl Acad Sci USA, 100(10):6081-6086 (May 13, 2003); US 2003-0138772, US 2007/0036760, US 2009/0197338, and WO 2010/138263, the contents of which relating to AAVs capsid proteins and associated nucleotide and amino acid sequences are incorporated herein by reference. Typically the methods involve culturing a host cell which contains a nucleic acid sequence encoding an AAV capsid protein or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the invention may be delivered to the packaging host cell using any appropriate genetic element (vector). The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAVs may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present invention include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the invention provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

In some aspects, the invention provides isolated cells. As used herein with respect to cell, the term “isolated” refers to a cell that has been isolated from its natural environment (e.g., from a tissue or subject). As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA) from a transcribed gene.

The foregoing methods for packaging recombinant vectors in desired AAV capsids to produce the rAAVs of the invention are not meant to be limiting and other suitable methods will be apparent to the skilled artisan.

### Recombinant AAV Vectors

The isolated nucleic acids (e.g., gene expression constructs) of the disclosure may be recombinant AAV vectors. The recombinant AAV vector may be packaged into a capsid protein and administered to a subject and/or delivered to a selected target cell. “Recombinant AAV (rAAV) vectors” are typically composed of, at a minimum, a transgene (e.g., an expression construct engineered to express a secreted gene product) and its regulatory sequences, and 5′ and 3′ AAV inverted terminal repeats (ITRs). The transgene may further comprise, as disclosed elsewhere herein, one or more regions that encode one or more inhibitory RNAs (e.g., miRNAs) comprising a nucleic acid that targets an endogenous mRNA of a subject.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

For nucleic acids encoding proteins, a polyadenylation sequence generally is inserted following the transgene sequences and before the 3′ AAV ITR sequence. A rAAV construct useful in the present invention may also contain an intron, desirably located between the promoter/enhancer sequence and the transgene. One possible intron sequence is derived from SV-40, and is referred to as the SV-40 T intron sequence. Any intron may be from the (3-Actin gene. Another vector element that may be used is an internal ribosome entry site (IRES).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, and the dihydrofolate reductase promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In another embodiment, the native promoter, or fragment thereof, for the transgene will be used. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken 3-actin promoter.

In some embodiments, one or more bindings sites for one or more of miRNAs are incorporated in a transgene of a rAAV vector, to inhibit the expression of the transgene in one or more tissues of a subject harboring the transgenes, e.g., non-liver tissues, non-lung tissues. The skilled artisan will appreciate that binding sites may be selected to control the expression of a transgene in a tissue specific manner. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

In some embodiments, the cloning capacity of the recombinant RNA vector may be limited and a desired coding sequence may involve the complete replacement of the virus's 4.8 kilobase genome. Large genes may, therefore, not be suitable for use in a standard recombinant AAV vector, in some cases. The skilled artisan will appreciate that options are available in the art for overcoming a limited coding capacity. For example, the AAV ITRs of two genomes can anneal to form head to tail concatamers, almost doubling the capacity of the vector. Insertion of splice sites allows for the removal of the ITRs from the transcript. Other options for overcoming a limited cloning capacity will be apparent to the skilled artisan.

### Recombinant AAV Compositions

The gene expression constructs (e.g., rAAVs comprising a gene expression construct) may be delivered to a subject in compositions according to any appropriate methods known in the art. In some embodiments, gene expression constructs are provided in a solution, comprising for example the gene expression construct (e.g., rAAV comprising the gene expression construct) suspended in a physiologically compatible carrier (e.g., a pharmaceutically acceptable excipient), and may be administered to a subject, e.g., a human, mouse, rat, cat, dog, sheep, rabbit, horse, cow, goat, pig, guinea pig, hamster, chicken, turkey, or a non-human primate (e.g., Macaque). The compositions of the invention may comprise a rAAV alone, or in combination with one or more other viruses (e.g., a second rAAV encoding having one or more different transgenes).

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Still others will be apparent to the skilled artisan.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration, the level of gene or RNA expression required to achieve a therapeutic effect, the specific disease or disorder being treated, and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. In some embodiments, a gene therapy construct (e.g., solution comprising a gene expression construct) is administered to a subject in a volume ranging from about 10 ml/kg to about 100 ml/kg (e.g., 10 ml/kg, 20 ml/kg, 30 ml/kg, 40 ml/kg, 50 ml/kg, 60 ml/kg, 70 ml/kg, 80 ml/kg, 90 ml/kg, or 100 ml/kg). In some embodiments, the volume of a solution is expressed as a percentage of a subjects lower extremity volume, for example, 10%, 20%, 30%, 40%, 50%, 60%, 70%, or 80% of a subject's lower extremity volume. In some cases, a dosage between about 10¹⁰ to 10¹² rAAV genome copies per subject is appropriate. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 .ANG., containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (ie., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only and the invention is described in detail by the claims that follow.

As used herein, the terms “approximately” or “about” in reference to a number are generally taken to include numbers that fall within a range of 1%, 5%, 10%, 15%, or 20% in either direction (greater than or less than) of the number unless otherwise stated or otherwise evident from the context (except where such number would be less than 0% or exceed 100% of a possible value).

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The entire contents of all references, publications, abstracts, and database entries cited in this specification are incorporated by reference herein.

### EXAMPLES

### Example 1: Materials and Methods

### Study Outline

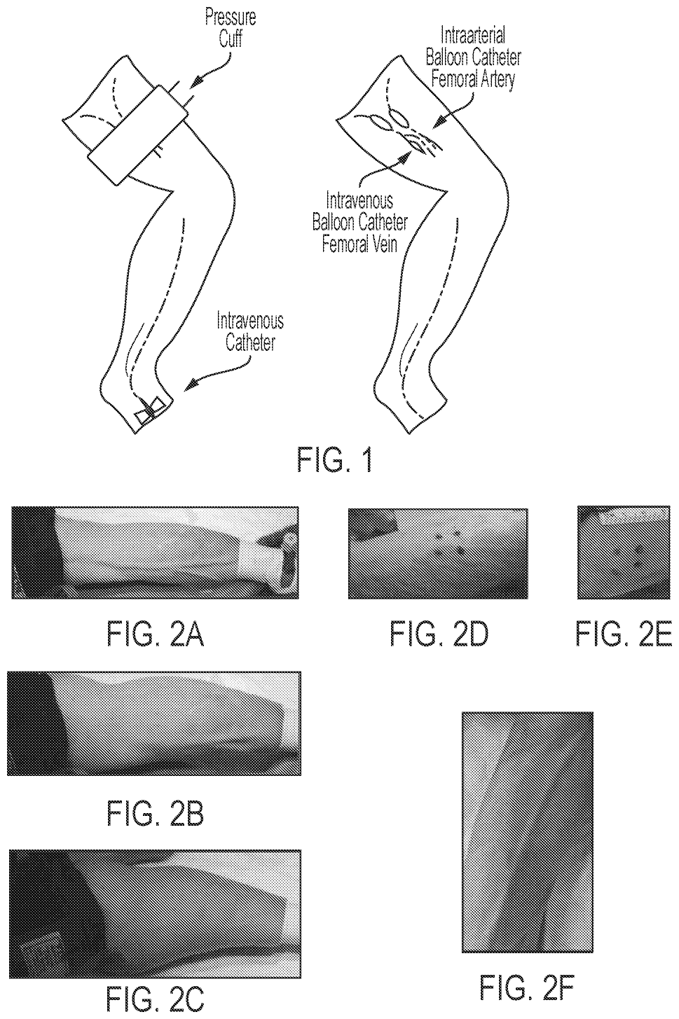

Animals were selected based on serum neutralizing antibodies against AAV1 or AAV8 as determined commercially using an in-vitro assay; animals with a titer at or below 1:10 were selected. Five groups of animals were administered rAAV1-CB-rhAATmyc or rAAV8-CB-rhAATmyc by intramuscular (IM) injection, intra-arterial push and dwell (IAPD), or venous limb perfusion (VLP) as assigned (Table 2) on study day 0. FIG. 1 is a schematic depicting VLP and IAPD procedures. The vector dose was 6×10¹² vg/kg for all groups. After dosing, animals were monitored by veterinary staff twice daily for 7 days for pain, bleeding, suture loss, limping, or other signs. Detailed clinical observations and body weight were recorded. At study day 60, animals were euthanized and subject to a complete necropsy and blood and tissues collected for evaluation.

TABLE 2
Study Design

| | | Test Article | | | |
|---|---|---|---|---|---|
| | | (6 × 10¹² | | | |
| Dose | Animal # | vg/kg for | | | |
| Group | (Sex; NAb titer) | all groups) | Route | Dosing volume | |
| IM - | RA1683 (F; 1:5) | rAAV1-CB- | IM | 0.5 mL/injection | |
| AAV1 | RA1598 (M; 1:10) | rhAATmyc | | (8 injections) ^{d} | |
| IAPD - | RA1709 (F; 1:5) | rAAV1-CB- | IAPD | 12.5 | mL/kg |
| AAV1 | RA1562 (M; 1:10) | rhAATmyc | | | |
| IAPD - | RA1660 (F; 1:10) | rAAV8-CB- | IAPD | 12.5 | mL/kg |
| AAV8 | RA1664 (F; 1:10) | rhAATmyc | | | |
| VLP - | RA0770 (F; <1:5) | rAAV1-CB- | VLP | 50 | mL/kg |
| AAV1 | RA1567 (M; 1:10) | rhAATmyc | | | |
| VLP - | RA1676 (F; 1:10) | rAAV8-CB- | VLP | 50 | mL/kg |
| AAV8 | RA1703 (F; 1:10) | rhAATmyc | | | |

### Vector Dosing

### Test Article Preparation

The vector was administered in volumes dictated by the injection or infusion procedure (Table 1). For each administration route, individual stock vials of vector were thawed and diluted on the day of use in the appropriate concentration and volume to deliver the targeted vector dose (6×10¹² vg/kg). The vector was diluted with Lactated Ringer's Solution.

### Intramuscular (IM) Injection

Animals were anesthetized with ketamine (10 mg/kg with 2-3 mg/kg bumps as needed) administered intramuscularly. For the IM dose group, rAAV1-CB-rhAATmyc vector was administered as eight, 0.5 mL injections (i.e., 4 mL of total dose volume), with the concentration adjusted to achieve the desired total dose based on the body weight of an animal. The injections were performed into the quadriceps and gastrocnemius muscles in the right hind limb with 4 injections in each muscle. The spacing between injections depended on the size of the muscle, but were 0.5 to 1 cm apart. The injection sights were marked with a black marking pen for photography of the injected limbs. Post injection pain, if observed, was managed with buprenorphine (0.01-0.03 mg/kg) administered IM. Thereafter, buprenorphine (0.01 to 0.03 mg/kg, IM) was administered as needed, based on clinical observations.

### Intravascular Limb Infusion

### Pre-surgical Preparation and Anesthesia

Aseptic technique was used throughout the surgical procedure for the IAPD and VLP delivery. For surgical procedures animals were pre-medicated with ketamine (10 mg/kg) administered intramuscularly. Inhalant anesthesia (generally 1-4% isoflurane in oxygen for induction and 0.5% to 3% isoflurane in oxygen as needed for maintenance) were administered via face mask to facilitate intubation. During the operative procedure anesthesia was maintained with 0.5% to 3% isoflurane in oxygen administered via the endotracheal tube.

One or two venous catheters were placed in a peripheral vein in the leg or arm (not the leg for the infusion). The catheters were used, as needed, to inject heparin and protamine, to withdraw blood for assessment of clotting time, to provide Plasmalyte (5 mL/kg/hr) during the infusion procedure.

### Intra-Arterial Push and Dwell (IAPD)

IAPD animals received the vector (rAAV1-CB-rhAATmyc or rAAV8-CB-rhAATmyc) in a volume of 12.5 mL/kg of Lactated Ringer's Solution. Buprenorphine (0.01 to 0.03 mg/kg, administered IM) was given preemptively at least 20 minutes prior to incising skin. The surgical site was prepared according to standard sterile procedure. After lidocaine (1 mg/kg) and bupivacaine (1 mg/kg) were administered by local application at the incision site, an incision was made in the lower anterior thigh of the right pelvic limb and the superficial femoral artery and vein dissected and isolated with silk suture. Arterial and venous access was obtained with sheath catheters. The catheters were inserted by cut-down and then retrograde positioned into upper femoral artery and vein near the inguinal ligament. The arterial and venous balloon catheters were then placed through their respective sheathes. The stopcock on the venous catheter was turned to prevent venous outflow. Correct placement of the catheters was checked by fluoroscopy, confirming the presence of the arterial and venous balloons above the level of the vascular branches leading to the quadriceps muscle groups. Once the vein and artery were cannulated, heparin was administered to achieve an activated clotting time of >350 sec determined using an i-STAT clinical analyzer and an activated clotting time (ACT) cartridge. The limb was elevated and wrapped tightly to massage all venous blood from the limb, after which the catheter balloons were inflated to prevent the vascular flow of the femoral vein and artery. The limb was then lowered and unwrapped. After a pre-flush with LRS (5 ml/kg), the vector (rAAV1-CB-rhAATmyc or rAAV8-CB-rhAATmyc) in a volume of 12.5 mL/kg was infused as quickly as possible though the arterial catheter sheath port. The vector solution was allowed to dwell for 15 minutes after which repeat fluoroscopy confirmed that the balloons had remained inflated through the entire dwell time. At that point a post-flush of 5 ml/kg of LRS will be injected into the arterial catheter. After the infusion had been completed, the balloons were deflated and catheters removed. The effects of the circulating heparin were reversed by injection of protamine (0.5-1 mg/100 USP heparin units administered). Blood samples were obtained and clotting time checked. When the clotting time had returned to near baseline value (±20 seconds), the animal was allowed to recover from anesthesia and returned to its home cage.

### Venous Limb Perfusion (VLP)

VLP dosed animals received the vector (rAAV1-CB-rhAATmyc or rAAV8-CB-rhAATmyc for Group 5) in a volume of 50 mL/kg of Lactated Ringer's Solution. For the VLP procedure, an intravenous catheter was placed into the distal peripheral saphenous vein of the right pelvic limb. The limb was elevated and wrapped tightly from distal to proximal (from just above catheter to mid-thigh) to massage as much blood as possible from the limb. A tourniquet was then placed around the level of the proximal thigh and tightened to prevent vascular flow into and out of the limb. The tourniquet extended from proximal to mid-thigh. The limb was then lowered and unwrapped. The vector (rAAV1-CB-rhAATmyc or rAAV8-CB-rhAATmyc) in a volume of 50 ml/kg was infused over about 5-10 minutes. The tourniquet remained tight for 15 minutes following the infusion and was then released. The catheter was removed and the animal allowed to recover from anesthesia and returned to its home cage.

### Physiological Parameter Monitoring During Infusions

Heart rate, respiratory rate and body temperature were monitored and documented during the surgical procedure to evaluate the status of animals.

### Post Vector Administration Monitoring and Observations

After vector administration on the dosing day, animals subjected to infusion procedures (Groups 2-5) were observed for evidence of erythema and edema of the infused site, blood vessel rupture, compartment syndrome, traumatic rhabodomyolysis, high intravascular pressure, bleeding (hematoma), pain, abnormal gait limping, potential damage to nerves, muscles or the vascular network.

In addition, after vector administration all animals (Groups 1-5) were monitored for clinical signs twice daily for 7 days. Behavioral and clinical observations were made on awake animals, with special attention paid to the legs and any abnormal motor movements (including posture or gait abnormalities).

For serum chemistry analyses, blood was collected into a serum separator or clot tubes for centrifugation to separate cellular and serum fractions. Serum chemistry was determined using a Hitachi Modular Analytics Clinical Chemistry System (Roche Diagnostics, Indianapolis, Ind.).

### Western Blot

Serum sample and standard were diluted in 1:50 PBS. 10 μl diluted serum were mixed with 10 ul of Tris-Glycine SDS sample buffer (2× Novex) heated at 85° C. for 10 min). 201 treated sample were run on Novex 12% Tris-Glycine gels (Invitrogen XP04125), USA) using Tris-Glycine SDS running buffer (Invitrogen, USA).

Protein was transferred to nitrocellulose membranes using an i-Blot transfer device (Invitrogen, USA). Membranes were blocked for 1 hour at room temperature with Odyssey Blocking Buffer (LiCor, USA) before being probed overnight with primary antibodies (1:1000 dilution) (goat cmyc antibody GenTex cat No. 30518). IR labeled secondary antibodies (1:5000 dilution) were applied (IRDye® 680LT Donkey anti-Goat IgG (H+L)). Blots were visualized using the Odyssey Infrared imaging system (LiCor, USA).

Images were processed using image studio program. Western blotting: all antibodies was used at the manufacturers recommended dilution.

Real-Time qRT-PCR

Frozen liver and gastrocnemius muscle samples from Day 60 were used to extract RNA using TRIzol Reagent. The RNA was then treated with a TURBO DNA-free Kit (Thermo Fisher Scientific, #AM1907) to remove DNA contamination before a high-capacity RNA-to-cDNA kit (Thermo Fisher Scientific, #4387406) was used for reverse transcription to obtain cDNAs. qPCR was subsequently performed using custom-designed Fam-labeled primers and probes targeting the transgene-c-myc junction (Thermo Fisher Scientific, #4448484). GAPDH was used as an endogenous control utilizing a VIC primer-limited expression assay (Thermo Fisher Scientific, #4451933).

### Genomic DNA Extraction and Real Time PCR

AAV genome copies were measured using qPCR. The tissues were harvested in a manner that prevented cross contamination, snap frozen in liquid nitrogen and stored at −80° C. until genomic DNA (gDNA) was extracted. gDNA was isolated from liver, right calf, left calf, right quadriceps, left quadriceps, right inguinal lymph node, left inguinal lymph node, cervical spinal cord, and lumbar spinal cord using a DNeasy blood and tissue kit (Qiagen, Valencia, Calif.) according to the manufacturer's instructions. gDNA concentrations were determined using the NanoDrop system (Thermo Fisher, Wilmington, Del.).

AAV genome copies present in gDNA were quantified by real-time PCR using the QuantStudio 3 Real-Time PCR System (Thermo Fisher, Carlsbad, Calif.—not actually sure of the location) according to the manufacturer's instructions, and results were analyzed using the QuantStudio Design & Analysis v1.4.1 software. Briefly, primers and probe were designed to the SV40 polyA region of the AAV vector used. A standard curve was performed using plasmid DNA containing the same SV40 pA target sequence. PCR reactions contained a total volume of 50 μl and were run at the following conditions: 50° C. for 2 minutes, 95° C. for 10 minutes, and 45 cycles of 95° C. for 15 seconds and 60° C. for 1 minute. DNA samples were assayed in triplicate. In order to assess PCR inhibition, the third replicate was spiked with plasmid DNA at a ratio of 100 copies/μg gDNA. If this replicate was greater than 40 copies/μg gDNA, then the results were considered acceptable. If a sample contained greater than or equal to 100 copies/μg gDNA, it was considered positive for vector genomes. If a sample contained less than 100 copies/μg gDNA, it was considered negative for vector genomes. Vector copy numbers reported are standardized per μg gDNA. Assay controls include: a No Template Control (NTC) with acceptability criteria <15 copies and an established study specific standard curve slope range (+/−3SD from three individual standard preparations and runs).

### IFNγ-ELISpot Response to AAV1 and AAV8 Capsids

Peripheral blood monocytes (PBMCs) were isolated before dosing and at day 60 post-injection and stimulated in vitro in R10 media supplemented with human IL-2 and IL-7 (1 ng/ml) and a complete set of AAV1 or AAV8 peptides (0.5 μg/ml) for 3 days. Then, cells were washed and resuspended in R10 media supplemented with human IL-2 and IL-7 (1 ng/ml) for 3 additional days. On day 6, cells were washed and left to rest overnight in R10 media. On day 7, the IFNγ-ELISpot assay was performed according to manufacturer's recommendations (Monkey IFNγ ELISpot^{BASIC}, MABTech). PBMCs were stimulated in vitro with overlapping peptides spanning the AAV1 or AAV8 capsid VP1 sequences, and divided into 3 pools (15-mers overlapping by 10 aa). A negative control consisted of unstimulated cells (medium only) whereas CD3/CD28 stimulation was used as a positive control for cytokine secretion.

### Example 2: Isolated Limb Perfusion Methods for rAAV Vector Delivery to Skeletal Muscle

### Limb Infusion Procedures

Animals were administered rAAV vectors as described in Example 1. FIGS. 2A-2F show photographs of limbs from injected animals. All animals tolerated both procedures well and recovered without incidence. The IAPD procedure had a total procedure time of around 4 hours and required three surgical personnel, one anesthetist, and two technical assistants to perform. The VLP procedure had a total procedure time of around 1 hour and required two technical assistants and one anesthetist to perform. The increased procedural time with the IAPD procedure resulted from the time to place the catheters surgically and the time to confirm catheter placement by fluoroscopy. Marked limb swelling was seen following the VLP procedure but this resolved completely within 12-24 hours post-procedure and did not alter the animal's ability to use the limb normally.

Serum c-Myc ELISA

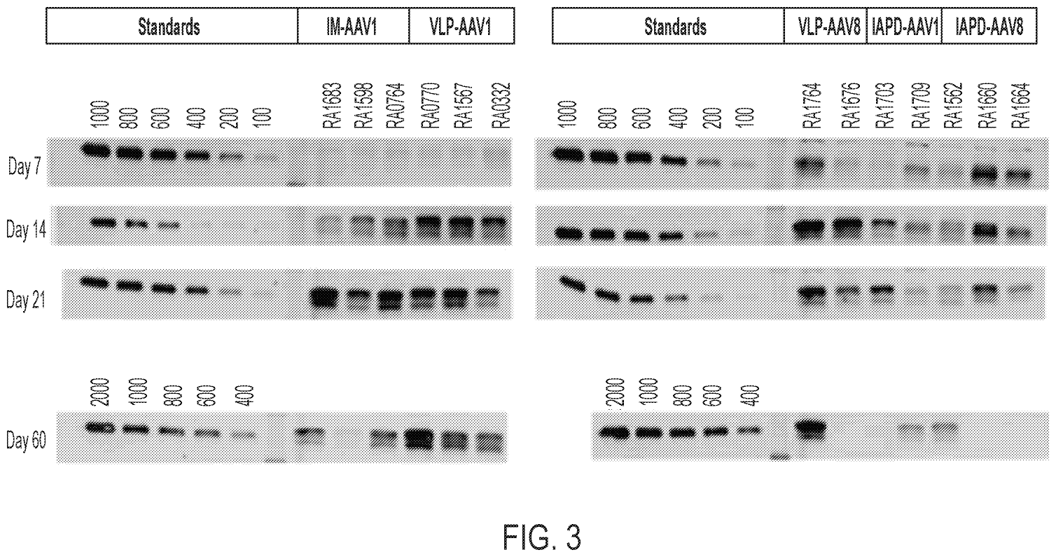

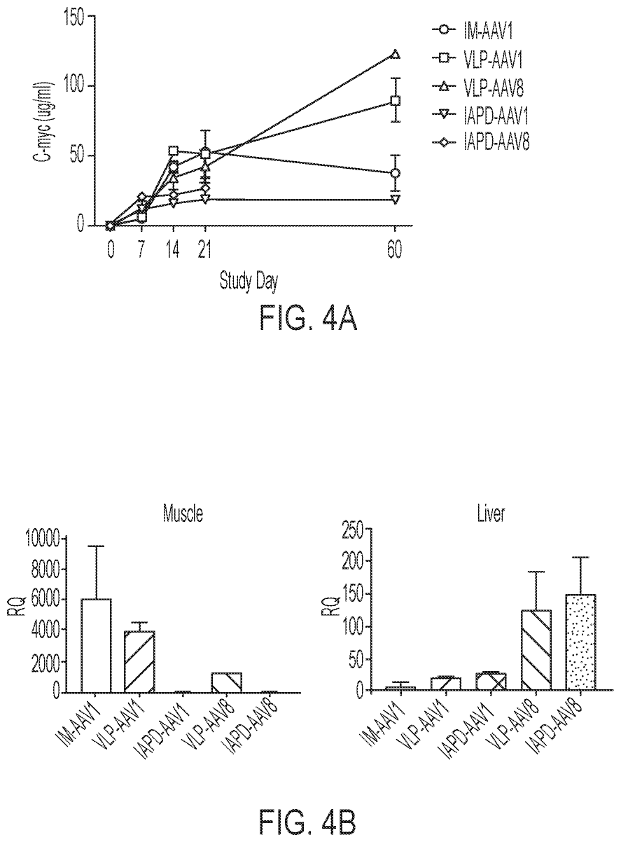

A myc-tag was included in the AAT transgene in order to allow monitoring of transgene expression without induction of an immune response in injected animals. Serum c-myc levels rise in all injection groups with both AAV1 and AAV8 capsids (FIGS. 3 and 4A). The AAV1 hydrodynamic group was observed to trend the highest.

Real-Time qRT-PCR

Primers targeting the AAT-c-myc junction were utilized to identify transgene RNA expression in the gastrocnemius muscle (from the site of injection in the IM-dosed animals) and liver at day 60 post-delivery. Muscle expression was higher in the IM and VLP groups compared to the IAPD groups (FIG. 4B). In the liver, RNA levels were highest in the VLP-AAV8 and IAPD-AAV8 groups. All AAV1 dosing groups had similar liver expression. Muscle expression was markedly higher than liver expression in all the AAV1 dosing groups.

### Serum Chemistry and Complete Blood Count

FIGS. 7A-7C show data relating to measurement of creatine kinase (CK), alanine transaminase (ALT) and aspartate transaminase (AST) in serum of injected animals. A moderate spike in serum creatine kinase (CK), a marker of muscle damage, was observed 1 day after IAPD vector delivery. A mild spike 21 days after AAV1 IAPD delivery was also observed. At Day 1 all other groups had minimal to no increase in serum creatine kinase. The IM group had a very mild increase in CK in one animal at Day 21. There was a moderate increase in serum ALT and AST at Day 1 post-delivery in the IAPD AAV1 group as well as a mild AST elevation at Day 21 in that same group. No other serum chemistry or complete blood counts that changed significantly were observed following vector dosing.

### IFNγ-ELISpot Response to AAV1 and AAV8 Capsids

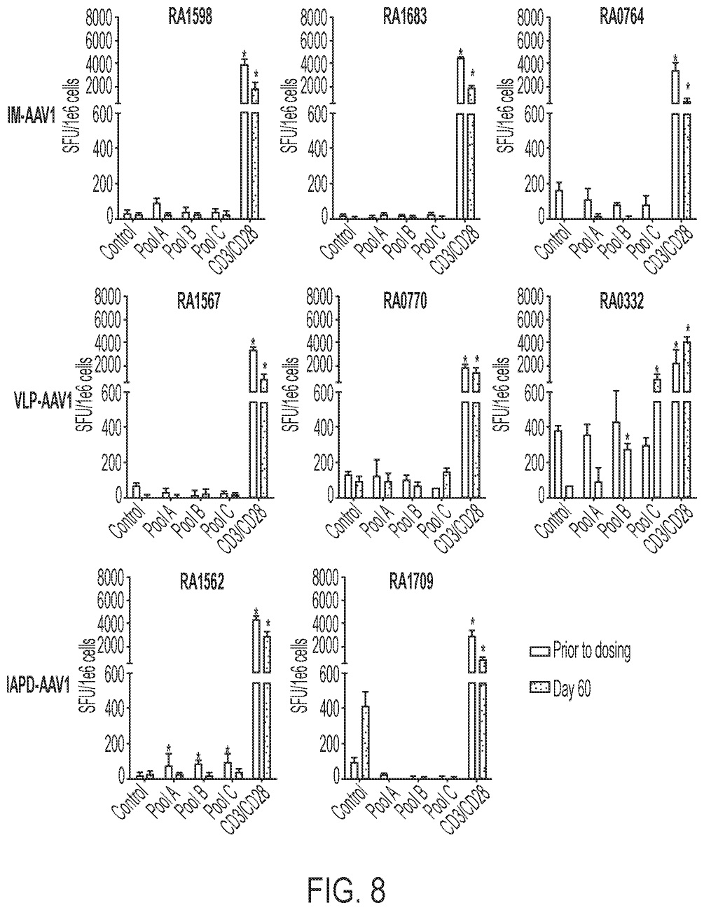

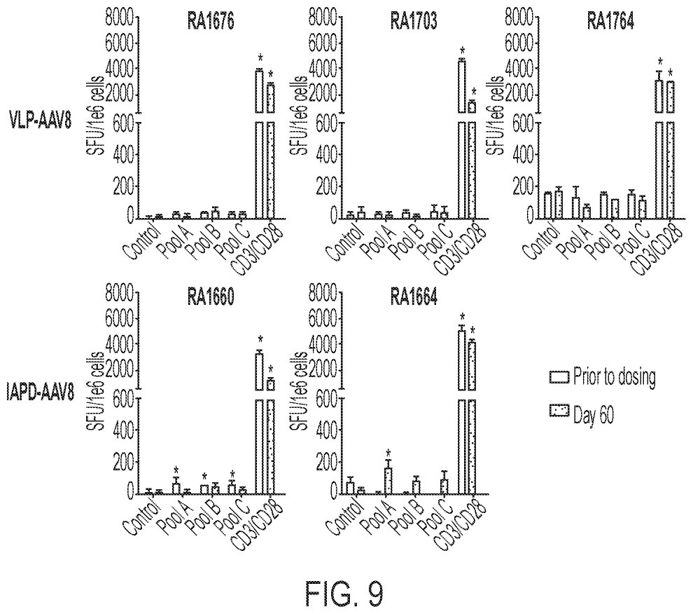

The T cell response to both AAV1 and AAV8 capsids were monitored by IFNγ ELSpot assay (Table 3 and FIGS. 8 and 9). Peripheral blood mononuclear cells (PBMC) were expanded for 6 days prior to the assay. Data indicate that none of the animals injected IM had a positive response prior to dosing and at Day 60 post-delivery. One animal injected IAPD had a positive response to AAV1 capsid prior to dosing but it was not confirmed at Day 60 post vector delivery. One out of 3 animals showed a mild positive response to AAV1 capsid at Day 60 post dosing (less than 350 spot forming unit (SFU) per million of cells).

None of the animals injected with the AAV8 vector via VLP had an IFNγ positive response to the capsid prior to and post dosing. One animal injected IAPD showed a positive response prior to dosing but was not confirmed at necropsy and the second animal had a mild positive response at necropsy (less than 200 SFU per million of cells). Data indicate there is no systematic cellular immune response to both AAV1 or AAV8 capsids after IM, VLP or IAPD vector administration.

TABLE 3

IFNγ secretion to AAV capsid

| Dose Group | Animal # | Prior to dosing | Day 60 |
|---|---|---|---|
| IM - AAV1 | RA1598 | − | − |
| | RA1683 | − | − |
| | RA0764 | − | − |
| VLP - AAV1 | RA1567 | − | − |
| | RA0770 | − | − |
| | RA0332 | − | + |
| IAPD - AAV1 | RA1562 | + | − |
| | RA1709 | − | − |
| VLP - AAV8 | RA1676 | − | − |
| | RA1703 | − | − |
| | RA1764 | − | − |
| IAPD - AAV8 | RA1660 | + | − |
| | RA1664 | − | + |

### Safety and Clinical Observations

Both limb infusion techniques were tolerated well by the animals. The hydrodynamic delivery was technically easier to perform because it did not require accessing the femoral artery and vein but rather just simple placement of the peripheral vein catheter, and it resulted in little or no muscle injury as indicated by the serum CK (muscle serum enzyme levels) in that group.

### Comparison of Total Vector Genomes Delivered to Muscle Via Various Modes of Delivery

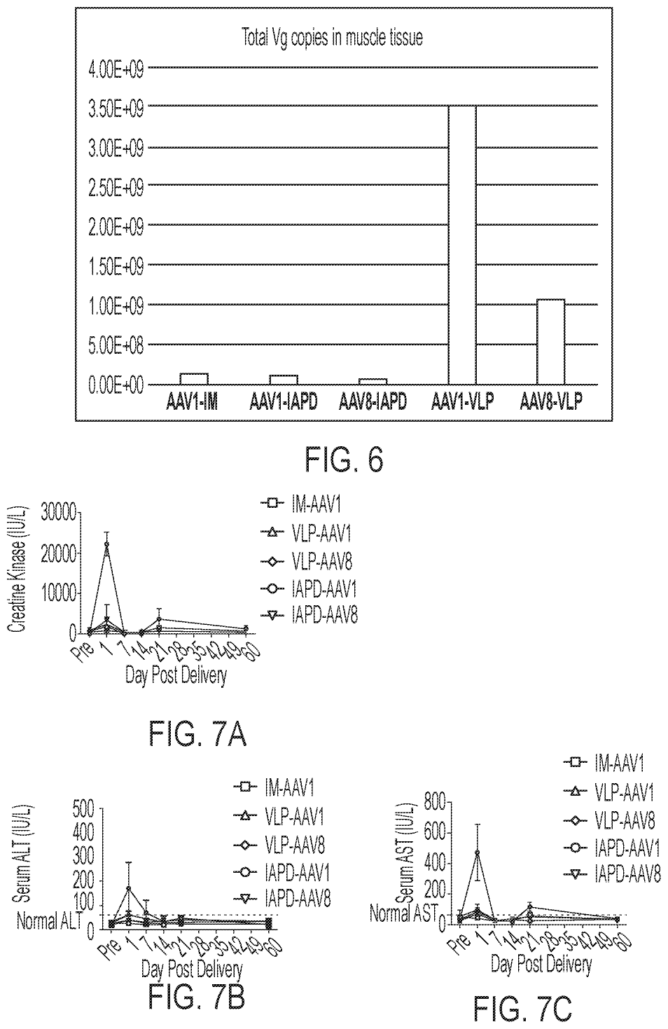

In order to confirm that the increased expression observed in the rAAV1-VLP group was due to an increase in the total number of vector genomes delivered to the lower extremity musculature, quantitative PCR for rAAV vector genomes, normalized to the quantity of genomic DNA (i.e., mcg of gDNA), was performed (FIG. 6). The volume of muscle transduced was determined using the estimated volume of the limb perfused by the vessel cannulated. In the case of IM, the volume of injection was used as an estimate of the volume of muscle tissue transduced, based on prior studies including real-time ultrasound performed during deltoid muscle injections in humans in a previous trial (e.g., as described in Brantly et al., Hum Gene Ther. 2006 December; 17(12):11′77-86.). The number of myofiber nuclei comprising that volume was then determined using an estimate of nuclear density (e.g., as described by Brusgaard, et al. Number and spatial distribution of nuclei in the muscle fibers of normal mice studied in vivo. Journ of Physiol 2003: 551.2; 467-478.). The number of vg copies delivered to the muscle was estimated at 25 times greater with rAAV1-VLP than rAAV1-IM.

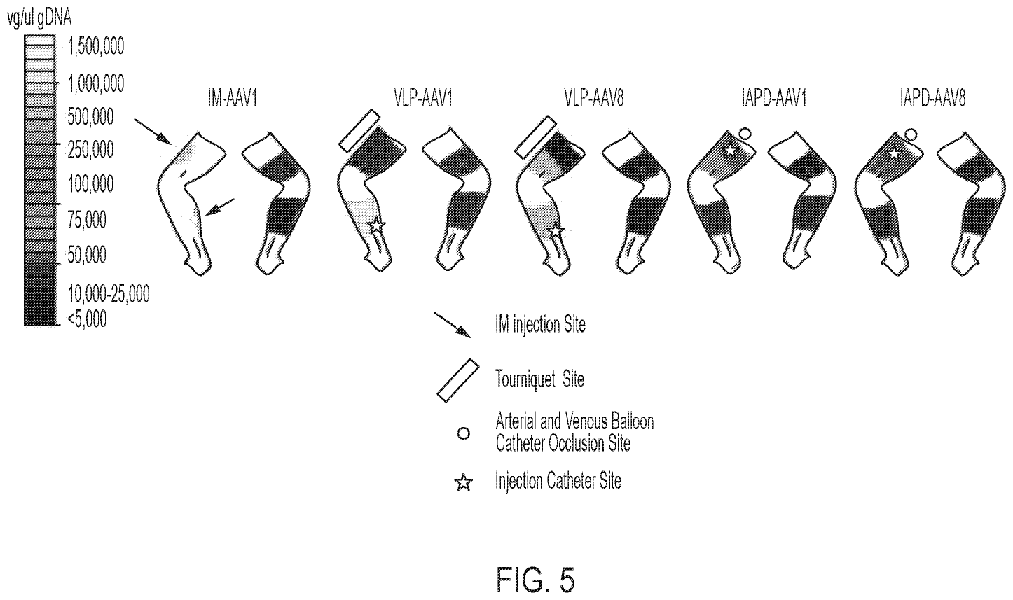

Next, the total number of vector genomes retained within the muscle (as described above) was compared with the total number of vector genomes detected in the liver, assuming that the liver of a rhesus macaque contains approximately 4.5×10¹⁰ nuclei. These data were then used to calculate the ratio (as a percentage) of the total vector genomes detected within the muscle, as compared with the total vector genomes detected within the liver, expressed as a percentage (muscle vg/liver vg×100), as shown in Table 4. A heatmap of vector genome distribution is shown in FIG. 5.

TABLE 4
Tissues (vector genomes/μg gDNA)

| | Right Upper | | Right Lower | | | | Right Inguinal | |
|---|---|---|---|---|---|---|---|---|
| | Quadriceps | | Quadriceps | | Right Calf | | Lymph Node | |
| Serotype- | (Dosed Limb) | | (Dosed Limb) | | (Dosed Limb) | | (Dosed Limb) | |
| Route | Mean | STD | Mean | STD | Mean | STD | Mean | STD |
| AAV1-IM | 498,778 | 281,500 | 574,208 | 562,329 | 1,599,935 | 1,115,968 | 2,532,248 | 1,319,764 |
| AAV1-IAPD | 44,150 | 37,218 | 129,055 | 127,706 | 22,530 | 18,723 | 368,133 | 111,128 |
| AAV8-IAPD | 86,205 | 91,689 | 70,038 | 25,065 | 16,885 | 8,792 | 293,298 | 103,261 |
| AAV1-VLP | 10,188 | 8,071 | 9,045 | 5,069 | 783,635 | 87,715 | 2,213,788 | 90,284 |
| AAV8-VLP | 1,775 | 526 | 203,595 | 121,972 | 239,328 | 84,476 | 980,443 | 19,295 |

| | | | | | | | Left Inguinal | |
|---|---|---|---|---|---|---|---|---|
| | | | Left Quadriceps | | Left Calf | | Lymph Node | |
| Serotype- | Liver | | (Undosed Limb) | | (Undosed Limb) | | (Undosed Limb) | |
| Route | Mean | STD | Mean | STD | Mean | STD | Mean | STD |
| AAV1-IM | 376,475 | 387,624 | 9,878 | 11,388 | 993 | 1,118 | 37,998 | 30,675 |
| AAV1-IAPD | 1,192,300 | 284,074 | 3,008 | 2,120 | 9,150 | 5,185 | 35,348 | 25,648 |
| AAV8-IAPD | 2,878,100 | 1,665,077 | 3,028 | 2,099 | 4,253 | 2,648 | 55,638 | 47,087 |
| AAV1-VLP | 1,961,980 | 470,336 | 13,040 | 11,487 | 9,025 | 6,956 | 92,483 | 39,652 |
| AAV8-VLP | 6,534,063 | 5,428,745 | 2,070 | 319 | 5,353 | 4,253 | 131,755 | — |

Interestingly, with direct IM injection of rAAV1-AAT, the total number of vg detected in the liver as a whole was calculated at 6.0×10¹⁰ vg, which is substantially greater than the amount retained within the muscle, which was 1.37×10⁸ vg. While rAAV-VLP did result in a 3-fold increase in total vg within the liver (up to 1.9×10¹¹ vg), the proportional increase retained in the muscle was much greater at over 25-fold (3.5×10⁹ vg as compared with 1.37×10⁸ vg). Comparing the ratio of total vg in muscle as compared with liver, rAAV1-IM showed muscle vg represented at only 0.22% of the abundance in liver, while rAAV1-VLP showed muscle vg at 1.09% of the total detected in liver. This represents a 5-fold increase in relative vg retention in muscle as compared with liver.

Quantitative PCR of vg genomes per mcg of DNA can be compared directly, since any vector hematogenously disseminated to the liver would likely have a similar distribution whether it spread from an IM or a limb perfusion source. As shown in Table 5, the rAAV1-IM group showed the least liver spread, while rAAV8-VLP showed the most. This observation is consistent with the known enhanced tropism of rAAV8 for the liver. The increase in vg in the liver comparing rAAV1-M to rAAV1-VLP is only 5.2-fold, while the increase in vg in the muscle in the same comparison is approximately 25-fold.

TABLE 5

| | | | | | | | Vector |
|---|---|---|---|---|---|---|---|
| | | | Muscle | | Number of | Total | genome |
| | Liver | Total | (copies | | nuclei within | number of | ratio |
| | (copies per | number of | per mcg | | the transduced | vector | (total |
| | mcg DNA ~ | vector | DNA ~ | Volume of | volume (2.5e6 | copies | muscle |
| | copies | genome | copies | distribution | nuclei per ml | in the | vs total |
| Vector | per 2.8e5 | copies | per 2.8e5 | within the | of muscle | transduced | liver ×100) |
| Route | nuclei) | in liver | nuclei) | muscle | tissue¹) | muscle | as Percent |
| AAV1 | 376,475 | 6.0e10 | 1,599,935 | 9.6 ml | 2.40e7 | 1.37e8 | 0.22% |
| IM | | copies | | | nuclei | copies | |
| AAV1- | 1,192,300 | 1.9e11 | 22,530 | 600 ml | 1.50e9 | 1.21e8 | 0.064% |
| IAPD | | copies | | | nuclei | | |
| AAV8- | 2,878,100 | 4.6e11 | 16,885 | 600 ml | 1.50e9 | 9.04e7 | 0.019% |
| IAPD | | copies | | | nuclei | | |
| AAV1- | 1,961,980 | 3.2e11 | 783,635 | 500 ml | 1.25e9 | 3.50e9 | 1.09% |
| VLP | | copies | | | nuclei | | |
| AAV8- | 6,534,063 | 1.1e12 | 239,328 | 500 ml | 1.25e9 | 1.07e9 | 0.15% |
| VLP | | copies | | | nuclei | | |

1. A method for delivering a transgene to a subject, the method comprising delivering a gene expression construct engineered to express one or more secreted gene products to an isolated limb of a subject, wherein circulation of blood through the vasculature of the isolated limb is interrupted, and wherein the delivery comprises the step of infusing a solution comprising the gene expression construct into a vein of the isolated limb.

2. The method of claim 1, wherein the gene expression construct comprises a viral vector.

3. The method of claim 2, wherein the viral vector is a recombinant adeno-associated virus (AAV) vector, adenoviral (Ad), lentiviral vector (LV), or retroviral vector.

4. The method of claim 2, wherein the viral vector is an rAAV vector.

5. The method of claim 1, wherein the secreted gene product is an Alpha-1 antitrypsin (AAT) protein.

6. The method of claim 5, wherein the AAT protein is non-human primate AAT.

7. The method of claim 5, wherein the AAT is a human AAT.

8. The method of claim 1, wherein the gene expression construct comprises an isolated nucleic acid encoding the secreted gene product.

9. The method of claim 1, wherein the subject is a mammal.

10. The method of claim 1, wherein the isolated limb is a lower extremity.

11. The method of claim 1, wherein the volume of the solution injected into the vein of the subject is between 10% and 50% of the lower extremity volume of the subject.

12. The method of claim 2, wherein between 1×10¹¹ and 1×10¹⁴ genome copies of the viral vector are delivered to the subject.

13. The method of claim 1, wherein the delivery of the gene expression construct occurs over a period of between 5 minutes and 120 minutes.

14. The method of claim 8, wherein the isolated nucleic acid sequence is operably linked to a promoter.

15. The method of claim 1, wherein the subject is a human.


SEQUENCE LISTING (2 sequences)

SEQ ID NO: 1 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - RNA, 3300 nt, Homo sapiens
ugggcaggaacugggcacugugcccagggcaugcacugccuccacgcagcaacccucagaguccugagcugaaccaagaaggaggagggggucgggccuccgaggaaggccuagccgcugcugcugccaggaauuccagguuggaggggcggcaaccuccugccagccuucaggccacucuccugugccugccagaagagacagagcuugaggagagcuugaggagagcaggaaagccucccccguugccccucuggauccacugcuuaaauacggacgaggacagggcccugucuccucagcuucaggcaccaccacugaccugggacagugaaucgacaaugccgucuucugucucguggggcauccuccugcuggcaggccugugcugccuggucccugucucccuggcugaggauccccagggagaugcugcccagaagacagauacaucccaccaugaucaggaucacccaaccuucaacaagaucacccccaaccuggcugaguucgccuucagccuauaccgccagcuggcacaccaguccaacagcaccaauaucuucuucuccccagugagcaucgcuacagccuuugcaaugcucucccuggggaccaaggcugacacucacgaugaaauccuggagggccugaauuucaaccucacggagauuccggaggcucagauccaugaaggcuuccaggaacuccuccguacccucaaccagccagacagccagcuccagcugaccaccggcaauggccuguuccucagcgagggccugaagcuaguggauaaguuuuuggaggauguuaaaaaguuguaccacucagaagccuucacugucaacuucggggacaccgaagaggccaagaaacagaucaacgauuacguggagaaggguacucaagggaaaauuguggauuuggucaaggagcuugacagagacacaguuuuugcucuggugaauuacaucuucuuuaaaggcaaaugggagagacccuuugaagucaaggacaccgaggaagaggacuuccacguggaccaggugaccaccgugaaggugccuaugaugaagcguuuaggcauguuuaacauccagcacuguaagaagcuguccagcugggugcugcugaugaaauaccugggcaaugccaccgccaucuucuuccugccugaugaggggaaacuacagcaccuggaaaaugaacucacccacgauaucaucaccaaguuccuggaaaaugaagacagaaggucugccagcuuacauuuacccaaacuguccauuacuggaaccuaugaucugaagagcguccugggucaacugggcaucacuaaggucuucagcaauggggcugaccucuccggggucacagaggaggcaccccugaagcucuccaaggccgugcauaaggcugugcugaccaucgacgagaaagggacugaagcugcuggggccauguuuuuagaggccauacccaugucuaucccccccgaggucaaguucaacaaacccuuugucuucuuaaugauugaacaaaauaccaagucuccccucuucaugggaaaaguggugaaucccacccaaaaauaacugccucucgcuccucaaccccuccccuccaucccuggcccccucccuggaugacauuaaagaaggguugagcuggucccugccugcaugugacuguaaaucccucccauguuuucucugagucucccuuugccugcugaggcuguaugugggcuccagguaacagugcugucuucgggcccccugaacuguguucauggagcaucuggcuggguaggcacaugcugggcuugaauccaggggggacugaauccucagcuuacggaccugggcccaucuguuucuggagggcuccagucuuccuuguccugucuuggaguccccaagaaggaaucacaggggaggaaccagauaccagccaugaccccaggcuccaccaagcaucuucaugucccccugcucaucccccacucccccccacccagaguugcucauccugccagggcuggcugugcccaccccaaggcugcccuccugggggccccagaacugccugaucgugccguggcccaguuuuguggcaucugcagcaacacaagagagaggacaauguccuccucuugacccgcugucaccuaaccagacucgggcccugcaccucucaggcacuucuggaaaaugacugaggcagauucuuccugaagcccauucuccauggggcaacaaggacaccuauucuguccuuguccuuccaucgcugccccagaaagccucacauaucuccguuuagaaucaggucccuucuccccagaugaagaggagggucucugcuuuguuuucucuaucuccuccucagacuugaccaggcccagcaggccccagaagaccauuacccuauaucccuucuccucccuagucacauggccauaggccugcugauggcucaggaaggccauugcaaggacuccucagcuaugggagaggaagcacaucacccauugacccccgcaaccccucccuuuccuccucugagucccgacuggggccacaugcagccugacuucuuugugccuguugcugucccugcagucuucagagggccaccgcagcuccagugccacggcaggaggcuguuccugaauagccccugugguaagggccaggagaguccuuccauccuccaaggcccugcuaaaggacacagcagccaggaaguccccugggccccuagcugaaggacagccugcucccuccgucucuaccaggaauggccuuguccuauggaaggcacugccccaucccaaacuaaucuaggaaucacugucuaaccacucacugucaugaauguguacuuaaaggaugagguugagucauaccaaauagugauuucgauaguucaaaauggugaaauuagcaauucuacaugauucagucuaaucaauggauaccgacuguuucccacacaagucuccuguucucuuaagcuuacucacugacagccuuucacucuccacaaauacauuaaagauauggccaucaccaagcccccuaggaugacaccagaccugagagucugaagaccuggauccaaguucugacuuuucccccugacagcugugugaccuucgugaagucgccaaaccucucugagccccagucauugcuaguaagaccugccuuugaguugguaugauguucaaguuagauaacaaaauguuuauacccauuagaacagagaauaaauagaacuacauuucuugca